(19) United States
(12) Patent Application Publication
(10) Pub. No.: US 2026/0188447 A1
(43) Pub. Date: Jul. 2, 2026

(54) SYSTEMS AND METHODS WITH ENSEMBLE WORD EMBEDDING AND TABULAR FEATURE DATASET FOR IMPROVED CONDITION MODEL PERFORMANCE

(71) Applicant: Iodine Software, LLC, Austin, TX (US)

(72) Inventor: Greg Hennigan, Round Rock, TX (US)

(21) Appl. No.: 19/439,063

(22) Filed: Jan. 2, 2026

Related U.S. Application Data
(60) Provisional application No. 63/741,240, filed on Jan. 2, 2025.

(51) Int. Cl. G16H 10/60; G06F 40/284; G06N 3/0464; G06N 3/08; G06V 30/414; G16H 50/30

(52) U.S. Cl. CPC G16H 10/60; G06F 40/284; G06N 3/0464; G06N 3/08; G06V 30/414; G16H 50/30

(57) ABSTRACT

Text data describing a patient's visit to a healthcare facility is extracted from a document and processed to remove stop words. Then, words associated with a medical condition can be determined and filtered to remove fully documented phrases that describe the medical condition. The filtered words are cleansed, ordered, and used to generate a final data set, which is used to train a first machine learning model to produce a numerical value that indicates a likelihood that the patient's visit to the healthcare facility is related to the medical condition. Features particular to the medical condition have feature attributes derived from historical data including medications, services, and observations pertaining to the medical condition. A data structure containing the numerical value, the features, and the feature attributes is input to a second machine learning model for generating a patient-specific score as evidence of the medical condition found during the patient's visit.

### CROSS-REFERENCE TO RELATED APPLICATION(S)

This application claims a benefit of priority under 35 U.S.C. § 119 (e) from Provisional Application No. 63/741,240, filed Jan. 2, 2025, entitled “METHOD FOR USING AN ENSEMBLE WORD EMBEDDING AND TABULAR FEATURE DATASET TO IMPROVE CONDITION MODEL PERFORMANCE,” the entire disclosure of which is fully incorporated by reference herein for all purposes.

### TECHNICAL FIELD

This disclosure relates generally to data processing using machine learning. More particularly, this disclosure relates to a system, method, and computer program product for more accurately detecting conditions in patients through an ensemble of machine learning techniques.

### BACKGROUND OF THE RELATED ART

In the field of clinical documentation improvement (CDI) technology, a previous solution to predicting the evidence of a medical condition relied on extracting specific pieces of text that were defined in the Unified Medical Language System (UMLS) dictionary, applying an attribute to the text (e.g., historical mention, negated mention, etc.), converting the text into an UMLS code (e.g., converting the term “chest pain” into a corresponding UMLS code C0001234), and using the particular UMLS code as a feature in a tabular data format that was then fed into a Gradient Boosting Machine (GBM) model. This prior approach was based on the assumption that the text existed and provided very little semantic meaning in conjunction with other supporting text around the text in question.

In view of the foregoing, there is a need for innovations and improvements in CDI technology, particularly with respect to condition model performance in detecting conditions in patients. The invention disclosed herein can address this need and more.

### SUMMARY OF THE DISCLOSURE

Embodiments disclosed herein can address the aforementioned drawbacks and provide additional technical solutions and benefits. An object of the invention is to provide a new machine learning model that is built on an ensemble of machine learning models that work in concert and that, as a whole, improve the performance of a CDI evidence model by more accurately detecting conditions present in patients. A description of the CDI technology can be found in U.S. Pat. No. 11,423,356, entitled “HIGH FIDELITY CLINICAL DOCUMENTATION IMPROVEMENT (CDI) SMART SCORING SYSTEMS AND METHODS,” which is incorporated by reference herein.

In some embodiments, a system implementing the invention disclosed herein is operable to extract text data and tabular feature dataset from raw data (e.g., a document) that describes a patient's visit to a healthcare facility. The system processes the text data through a series of data transformations, including one that applies a self-generated word embedding model to find words and/or phrases that best describe the patient's visit to the healthcare facility. The words and/or phrases are further processed and ordered for use as input to a first machine learning model (e.g., a Convolutional Neural Network model). This ordering allows the first machine learning model to run faster. The first machine learning model, in turn, produces a numerical value that indicates a likelihood that the patient's visit to the healthcare facility is related to a medical condition of interest.

Further, the system processes the tabular feature dataset to derive features particular to the medical condition of interest. The system then populates a data structure with the features and corresponding values from the raw data. The output from the first machine learning model (i.e., the numerical value that indicates a likelihood that the patient's visit to the healthcare facility is related to the medical condition of interest) is also added to the data structure.

The data structure is provided as input to a second machine learning model (e.g., an Extreme Gradient Boosting model). The second machine learning model learns from data contained in the data structure and, based on the learned knowledge, generates a prediction on whether the medical condition of interest exists for the patient. Through this ensemble of machine learning models, combined with knowledge gleaned from the transformed text data and the tabular feature dataset, the system gains a greater understanding of the meaning of patient-related text and, therefore, can provide improved, more accurate detection of conditions in patients, even if these conditions were not well documented and, therefore, may not be detected by the prior approach discussed above.

In some embodiments, a method can comprise extracting, from a document, text data describing a patient's visit to a healthcare facility; removing stop words from the text data, wherein the removing produces clean text data; determining, using a data transformation model or a large language model, words associated with a medical condition; filtering, from the words, fully documented phrases that describe the medical condition, wherein the filtering produces a filtered list of words; arranging the filtered list of words in order, wherein the arranging produces an ordered set of words; generating, using the ordered set of words, a final data set; training a first machine learning model using the final data set, wherein the first machine learning model is trained to produce a numerical value that indicates a likelihood that the patient's visit to the healthcare facility is related to the medical condition; determining, in a pre-processing stage, features particular to the medical condition, each feature having feature attributes derived from historical data including medications, services, and observations pertaining to the medical condition; preparing a data structure containing the features and the feature attributes; adding the numerical value from the first machine learning model to the data structure; applying a second machine learning model on the data structure, wherein the second machine learning model learns from the features, the feature attributes, and the numerical value from the first machine learning model, and generates a patient-specific score indicating a probability of the patient having the medical condition; and storing the patient-specific score in a data store as evidence of the medical condition being found during the patient's visit to the healthcare facility.

In some embodiments, wherein the generating the final data set comprises: tokenizing the ordered set of words into corresponding numerical values; populating an array with the numerical values; and padding entries in the array that do not have any numerical values with zeros.

In some embodiments, the first machine learning model comprises a Convolutional Neural Network model and the second machine learning model comprises an Extreme Gradient Boosting model. In some embodiments, the first machine learning model comprises a binary classification model for text processing and the second machine learning model runs an Extreme Gradient Boosting algorithm.

In some embodiments, the large language model is configured for: extracting, from the document, document embeddings that describe the document; extracting, from the document, N-gram words; and determining, from the document embeddings and the N-gram words using a cosine similarity, the words.

In some embodiments, preparing the data structure comprises processing a plurality of tables using a configuration file which stores a list of what medications, services, and observations pertain to the medical condition. In some embodiments, the configuration file is one of a plurality of configuration files, each specific to a medical condition.

One embodiment comprises a system comprising a processor and a non-transitory computer-readable storage medium that stores computer instructions translatable by the processor to perform a method substantially as described herein. Another embodiment comprises a computer program product having a non-transitory computer-readable storage medium that stores computer instructions translatable by a processor to perform a method substantially as described herein. Numerous other embodiments are also possible.

These, and other, aspects of the disclosure will be better appreciated and understood when considered in conjunction with the following description and the accompanying drawings. It should be understood, however, that the following description, while indicating various embodiments of the disclosure and numerous specific details thereof, is given by way of illustration and not of limitation. Many substitutions, modifications, additions, and/or rearrangements may be made within the scope of the disclosure without departing from the spirit thereof, and the disclosure includes all such substitutions, modifications, additions, and/or rearrangements.

### BRIEF DESCRIPTION OF THE DRAWINGS

The drawings accompanying and forming part of this specification are included to depict certain aspects of the invention. A clearer impression of the invention, and of the components and operation of systems provided with the invention, will become more readily apparent by referring to the exemplary, and therefore non-limiting, embodiments illustrated in the drawings, wherein identical reference numerals designate the same components. Note that the features illustrated in the drawings are not necessarily drawn to scale.

FIG. 1 is a flow diagram that illustrates a process for training and using a new machine learning model that is built on an ensemble of machine learning models that can learn from particularly prepared text data as well as tabular data so as to more accurately detect presence of patient conditions, according to some embodiments disclosed herein.

FIG. 2 depicts a diagrammatic representation of a data processing system for implementing an embodiment disclosed herein.

### DETAILED DESCRIPTION

The invention and the various features and advantageous details thereof are explained more fully with reference to the non-limiting embodiments that are illustrated in the accompanying drawings and detailed in the following description. Descriptions of well-known starting materials, processing techniques, components and equipment are omitted so as not to unnecessarily obscure the invention in detail. It should be understood, however, that the detailed description and the specific examples, while indicating some embodiments of the invention, are given by way of illustration only and not by way of limitation. Various substitutions, modifications, additions, and/or rearrangements within the spirit and/or scope of the underlying inventive concept will become apparent to those skilled in the art from this disclosure.

Embodiments disclosed herein provide a new system, method, and computer program product for more accurately detecting patient conditions through a new machine learning model. The new machine learning model is trained to process particularly prepared different types of inputs (e.g., text data and tabular data) through an ensemble of machine learning models, including a first machine learning model for processing a first type of data (e.g., the text data) and a second machine learning model for processing a second type of data (e.g., a tabular feature dataset).

As a non-limiting example, the first machine learning model can be a Convolutional Neural Network (CNN) model and the second machine learning model can be an Extreme Gradient Boosting (XGBoost) model. A CNN (or ConvNet) is a network architecture for deep learning that learns directly from data. CNNs are useful for finding patterns in images to recognize objects, classes, and categories. XGBoost generally refers to a scalable, distributed gradient-boosted decision tree (GBDT) machine learning library. It provides parallel tree boosting and is the current leading machine learning library for regression, classification, and ranking problems.

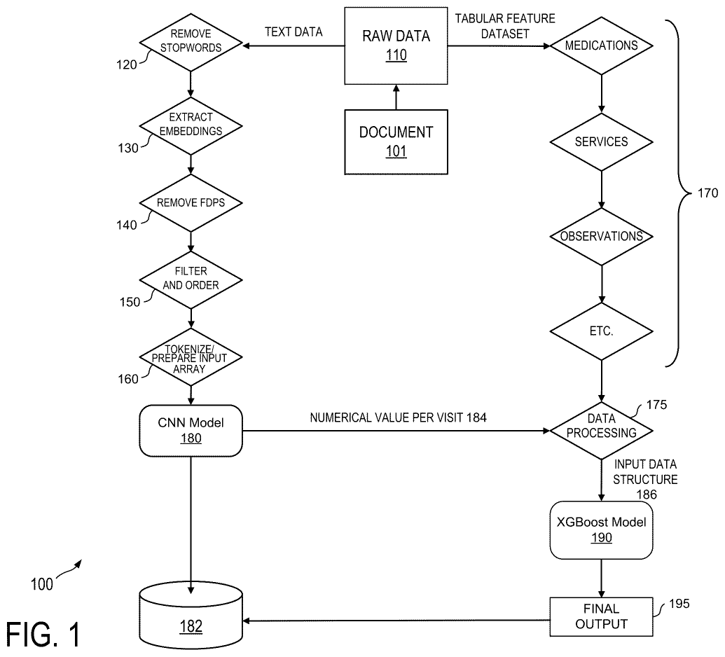

In some embodiments, the CNN model is particularly trained for text processing and the XGBoost model is configured for taking an input data structure that includes an output from the particularly trained CNN model. In this way, the new machine learning model takes inputs of two different types of data: text data (which is processed by the particularly trained CNN model) and tabular data (which is processed by the XGBoost model which takes an input data structure that includes an output from the particularly trained CNN model). The new machine learning model is further described below with reference to FIG. 1.

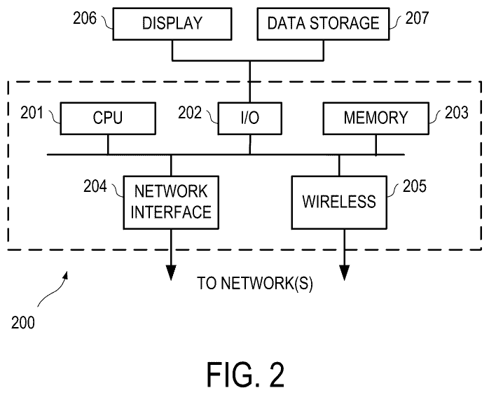

FIG. 1 is a flow diagram that illustrates a process 100 for training and using the new machine learning model that, as illustrated in the example of FIG. 1, includes an ensemble of various data transformation models, the CNN model, and the XGBoost model. In some embodiments, a data processing system or engine is configured for performing the process 100 on a computer (e.g., a server machine), an example of which is shown in FIG. 2.

As illustrated in FIG. 1, a document (e.g., a physician's note) 101 describing a patient's visit to a healthcare facility (e.g., a hospital, a clinic, etc.) provides a data source for raw data 110. In some embodiments, the raw data 110 can include historical data stored in tables (e.g., a table for medications for which patient during which visit, a table of what services performed for which patient during which visit, a table of observations of which patient during which visit, etc.). In embodiments disclosed herein, text data is extracted from the raw data 110 from the document 101.

### Text Data

Referring to FIG. 1, the text data thus extracted is kept in order and cleaned in a cleansing process 120 to remove stop words (i.e., words that are used frequently and that have little meaning to a medical condition of interest). This cleansing process produces clean text data.

Using a subset of cases (i.e., a training set) that have a particular medical condition of interest, the clean text data is run through a filtering process 130 that utilizes a keyword extraction tool (e.g., a Large Language Model (LLM) based tool, such as KeyBERT, for keyword extraction). In some embodiments, the keyword extraction tool leverages BERT embeddings and basic cosine similarity to determine a set of words (e.g., keyword phrases) that have the highest correlation to the positive case. KeyBERT refers to a minimal method for keyword extraction with BERT. The keyword extraction is done by finding sub-phrases in a document that are the most similar to the document itself. First, document embeddings are extracted with BERT to get a document-level representation. Then, word embeddings are extracted for N-gram words/phrases. Finally, cosine similarity can be used to find the words/phrases that are the most similar to the document. The most similar words could then be identified as the words that best describe the entire document.

Other transformer models can also be used. For instance, for a given medical condition, Acute Blood Loss Anemia, a transformer model can examine data and determine what beneficial related words are associated with this medical condition and extract them from patient records. In one embodiment, the patient records are from a single healthcare facility (e.g., a hospital) with a sample size of at least 250 patients or more. Further, a single transformer model can be configured for identifying words associated with multiple medical conditions. In some embodiments, there can be multiple transformer models, each configured for identifying words associated with a particular medical condition.

Next, fully documented phrases (“FDPs”) are removed, through another filtering process 140, from the list of words/phrases, resulting in a filtered list of words/phrases. These FDPs, which can be obvious medical terms used by physicians, provide the documentation for the patient's medical condition and need to be removed, so that the machine learning model can find only the evidence for the medical condition, rather than look at the FDPs to make a prediction.

The filtering process cleans the text again to remove even more unnecessary words/phrases and results in a list of words/phrases that should only be considered in the machine learning model (e.g., keywords for a particular medical condition, for instance, “impairments,” “pediatricians,” “postnatal,” “prediabetic,” “shoulder,” “complaint,” “concern,” “plantar,” “aphasia,” etc.). Then, the filtered list of keywords/phrases is processed once again, through yet another filtering process 150, so that these keywords/phrases are arranged in their order. This ordering process allows a very directed set of keywords/phrases to be used in the machine learning model. This directed set allows the machine learning model to run faster from a smaller set of data.

Finally, the directed/ordered set of keywords/phrases is tokenized and padded, through a tokenization process 160, to create a final data set. The tokenization process 160 may use any suitable tokenizer. As a non-limiting example, the final data set may take a form of an array as shown in Table 1 below.

TABLE 1
CNN Model Sample Input Data

| | 0 | 1 | 2 | . . . |
|---|---|---|---|---|
| 0 | 0 | 0 | 0 | . . . |
| 1 | 0 | 0 | 0 | . . . |
| 2 | 0 | 0 | 0 | . . . |
| 3 | 0 | 0 | 0 | . . . |
| 4 | 0 | 0 | 0 | . . . |
| 5 | 85 | 425 | 95 | . . . |
| 6 | 0 | 0 | 0 | . . . |
| 7 | 0 | 0 | 0 | . . . |
| . . . | . . . | . . . | . . . | . . . |

In Table 1, the entries from left to right follow the order of words from a sentence in the document 101. A non-limiting example of the document 101 can be a physician note by a document who wrote a sentence that describes a patient (e.g., “A patient came in with a high fever . . . ”).

The entries, from top to bottom, follow the order of the sentences in the document 101. Through the cleansing process 120, stop words not related to the medical condition of interest are removed. Through the filtering process 130, statistically relevant words and phrases are found. Through the filtering process 140, words that can serve as evidence of the medical condition are found (instead of words/phrases that document the medical condition of interest).

Further, through the filtering process 150, only words/phrases that are directly related to the medical condition are kept. These pre-processing steps allow a very efficient tokenization. For instance, suppose that the word “large” can be transformed into a numerical value of 85, the word “blood” can be transformed into a numerical value of 425, and the word “loss” can be transformed into a numerical value of 95, and further suppose that the fifth entry for the list of ordered words contains three words “large blood loss,” then the fifth entry can be tokenized into 85, 425, 95, respectively, as shown in Table 1 above. Since other entries at the same first, second, and third positions do not have words, they are padded with zeros.

This final dataset is used to train a binary classification model for text. Following the above example, the final dataset is used as input to train a CNN model 180, as shown in FIG. 1. The CNN model 180, in turn, produces a result for the patient associated with the document 101. Since the document 101 describes the patient's visit, the output from the CNN model 180 can be a numerical value that indicates a likelihood that the patient's visit to the healthcare facility is related to the medical condition of interest. The numerical value can be a number between 0 and 1. The CNN model 180 thus trained can then be stored in a data store 182 with the output.

### Tabular Data

Referring to FIG. 1, in a pre-processing stage 170, a set of features particular to a certain medical condition of interest is identified. These features can include, but are not limited to, lab results, medications, tests, cultures, etc.

Then, features attributes are configured around each feature thus identified. For instance, if hemoglobin is considered as a useful lab result, then hemoglobin-related feature attributes are determined, for instance, “minimum value,” “maximum value,” “number of times that the max value was above a given reference range,” and a number of other calculations.

As shown in FIG. 1, such feature attributes can derive from medications, services, observations, etc., and processed to prepare the feature data (in a tabular form) as input 186 to a final machine learning model 190 that, in this example, runs an XGBoost algorithm.

The text data output (i.e., a numerical value per a patient's visit) 184 from the CNN model 180 is now combined, through a data processing step 175, with the processed tabular feature data from the pre-processing stage 170 as inputs in a data structure 186 for the XGBoost model 190.

In some cases, at the pre-processing stage 170, the data processing engine may utilize historical data stored in tables (e.g., a table for medications, a table for services, a table for observations, etc.). The pre-processing engine takes these tables as input and processes them based on a configuration file which stores a list of what to process-what medications, what services, what observations, etc. There can be multiple configuration files, each specific to a particular medical condition.

The data processing engine processes the tables to extract features of interest from the tables (e.g., medications, services, observations, etc.) based on a configuration file for the medical condition of interest. The configuration file specifies what it needs (e.g., certain medical-condition-specific parameters/variables that need values) and the data processing engine calculates and uses the calculated values to determine a probability that the medical condition exists—i.e., a medical-condition-specific score, between 0 and 1, that the medical condition happened to the respective patient—each patient gets a score). The predictions can be stored in a new “results” table.

Table 2 below is a non-limiting example of an input data structure 186.

TABLE 2
XGBoost Model Sample Input Data

| visitId | result | docOutput | bloodpressuresystolic_initial | . . . |
|---|---|---|---|---|
| 8904730_hcagc | 0 | 0.000796 | 177 | . . . |
| 271711_ahn | 0 | 0.004077 | 111 | . . . |
| 5987424_tenetca | 0 | 0.001668 | 110 | . . . |
| 7377557_ukhs | 0 | 0.041904 | 157 | . . . |
| . . . | 0 | 0.001798 | 134 | . . . |

In the example of Table 2, the “docOutput” column is populated (e.g., through the data processing step 175) with the numerical value per visit output 184 from the transformer model 180. The XGBoost model 190 is operable to process the input data structure 186 and determine, for each visit, whether the patient's visit concerns a particular medical condition of interest. The results, each of which is a numerical value between 0 and 1, are used to update the input data structure 186, as shown in Table 2. The final output 195, a prediction on whether a medical condition of interest exists for a patient, can be stored in the data store 182.

As those skilled in the art can appreciate, predictions can be computationally expensive to make. Using a combination of a transformer model for keyword extraction and a tokenizer for transforming text values into numerical values, among various filtering processes, help to produce a streamlined input array to the CNN model 180. This array allows the CNN model 180 to run very efficiently (e.g., reducing 15,000 columns of data into 3000 columns of data).

Further, because the input array contains data highly relevant to a medical condition of interest, the CNN model 180 can perform better by producing more accurate predictions. Likewise, the pre-processing stage 170 identifies features found in many different types of inputs (e.g., lab results, medications, tests, cultures, etc.) that are associated with the same patient and that are particular to a certain medical condition of interest.

Combined with the per-visit predictions from the CNN model 180, the XGBoost model 190 can generate a patient-specific score indicating a probability of the patient having the particular medical condition. The new machine learning model disclosed herein can examine data points from many perspectives and look for evidence to validate what medical condition is documented (e.g., via the CDI technology described in the above-referenced U.S. Pat. No. 11,423,356).

FIG. 2 depicts a diagrammatic representation of a data processing system for implementing an embodiment disclosed herein. As shown in FIG. 2, data processing system 200 may include one or more central processing units (CPU) or processors 201 coupled to one or more user input/output (I/O) devices 202 and memory devices 203. Examples of I/O devices 202 may include, but are not limited to, keyboards, displays, monitors, touch screens, printers, electronic pointing devices (for example, mouse, trackball, stylus, touch pad, etc.), or the like.

Embodiments discussed herein can be implemented in a computer communicatively coupled to a network (for example, the Internet), another computer, or in a standalone computer. As is known to those skilled in the art, a suitable computer can include a central processing unit (“CPU”), at least one read-only memory (“ROM”), at least one random access memory (“RAM”), at least one hard drive (“HD”), and one or more input/output (“I/O”) device(s). The I/O devices can include a keyboard, monitor, printer, electronic pointing device

Examples of memory devices 203 may include, but are not limited to, hard drives (HDs), magnetic disk drives, optical disk drives, magnetic cassettes, tape drives, flash memory cards, random access memories (RAMs), read-only memories (ROMs), smart cards, etc. Data processing system 200 can be coupled to display 206, information device 207 and various peripheral devices (not shown), such as printers, plotters, speakers, etc. through I/O devices 202. Data processing system 200 may also be coupled to external computers or other devices through network interface 204, wireless transceiver 205, or other means that is coupled to a network such as a local area network (LAN), wide area network (WAN), or the Internet.

While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

ROM, RAM, and HD are computer memories for storing computer-executable instructions executable by the CPU or capable of being compiled or interpreted to be executable by the CPU. Suitable computer-executable instructions may reside on a computer-readable medium (e.g., ROM, RAM, and/or HD), hardware circuitry or the like, or any combination thereof. Within this disclosure, the term “computer-readable medium” is not limited to ROM, RAM, and HD and can include any type of data storage medium that can be read by a processor. For example, a computer-readable medium may refer to a data cartridge, a data backup magnetic tape, a floppy diskette, a flash memory drive, an optical data storage drive, a CD-ROM, ROM, RAM, HD, or the like. The processes described herein may be implemented in suitable computer-executable instructions that may reside on a computer-readable medium (for example, a disk, CD-ROM, a memory, etc.). Alternatively, the computer-executable instructions may be stored as software code components on a direct access storage device array, magnetic tape, floppy diskette, optical storage device, or other appropriate computer-readable medium or storage device.

Any suitable programming language can be used to implement the routines, methods or programs of embodiments of the invention described herein, including C, C++, Java®, JavaScript®, HTML, or any other programming or scripting code, etc. Other software/hardware/network architectures may be used. For example, the functions of the disclosed embodiments may be implemented on one computer or shared/distributed among two or more computers in or across a network. Communications between computers implementing embodiments can be accomplished using any electronic, optical, radio frequency signals, or other suitable methods and tools of communication in compliance with known network protocols.

Different programming techniques can be employed such as procedural or object oriented. Any particular routine can execute on a single computer processing device or multiple computer processing devices, a single computer processor or multiple computer processors. Data may be stored in a single storage medium or distributed through multiple storage mediums, and may reside in a single database or multiple databases (or other data storage techniques). Although the steps, operations, or computations may be presented in a specific order, this order may be changed in different embodiments. In some embodiments, to the extent multiple steps are shown as sequential in this specification, some combination of such steps in alternative embodiments may be performed at the same time. The sequence of operations described herein can be interrupted, suspended, or otherwise controlled by another process, such as an operating system, kernel, etc. The routines can operate in an operating system environment or as stand-alone routines. Functions, routines, methods, steps, and operations described herein can be performed in hardware, software, firmware, or any combination thereof.

Embodiments described herein can be implemented in the form of control logic in software or hardware or a combination of both. The control logic may be stored in an information storage medium, such as a computer-readable medium, as a plurality of instructions adapted to direct an information processing device to perform a set of steps disclosed in the various embodiments. Based on the disclosure and teachings provided herein, a person of ordinary skill in the art will appreciate other ways and/or methods to implement the invention.

It is also within the spirit and scope of the invention to implement in software programming or code an of the steps, operations, methods, routines or portions thereof described herein, where such software programming or code can be stored in a computer-readable medium and can be operated on by a processor to permit a computer to perform any of the steps, operations, methods, routines or portions thereof described herein. The invention may be implemented by using software programming or code in one or more digital computers, by using application specific integrated circuits, programmable logic devices, field programmable gate arrays, optical, chemical, biological, quantum or nanoengineered systems, components and mechanisms may be used. In general, the functions of the invention can be achieved by any means as is known in the art. For example, distributed, or networked systems, components and circuits can be used. In another example, communication or transfer (or otherwise moving from one place to another) of data may be wired, wireless, or by any other means.

A “computer-readable medium” may be any medium that can contain, store, communicate, propagate, or transport the program for use by or in connection with the instruction execution system, apparatus, system or device. The computer-readable medium can be, by way of example only but not by limitation, an electronic, magnetic, optical, electromagnetic, infrared, or semiconductor system, apparatus, system, device, propagation medium, or computer memory. Such computer-readable medium shall be machine readable and include software programming or code that can be human readable (e.g., source code) or machine readable (e.g., object code). Examples of non-transitory computer-readable media can include random access memories, read-only memories, hard drives, data cartridges, magnetic tapes, floppy diskettes, flash memory drives, optical data storage devices, compact-disc read-only memories, and other appropriate computer memories and data storage devices. In an illustrative embodiment, some or all of the software components may reside on a single server computer or on any combination of separate server computers. As one skilled in the art can appreciate, a computer program product implementing an embodiment disclosed herein may comprise one or more non-transitory computer-readable media storing computer instructions translatable by one or more processors in a computing environment.

A “processor” includes any hardware system, mechanism, or component that processes data, signals or other information. A processor can include a system with a central processing unit, multiple processing units, dedicated circuitry for achieving functionality, or other systems. Processing need not be limited to a geographic location, or have temporal limitations. For example, a processor can perform its functions in “real-time,” “offline,” in a “batch mode,” etc. Portions of processing can be performed at different times and at different locations, by different (or the same) processing systems.

It will also be appreciated that one or more of the elements depicted in the drawings/figures can also be implemented in a more separated or integrated manner, or even removed or rendered as inoperable in certain cases, as is useful in accordance with a particular application. Additionally, any signal arrows in the drawings/figures should be considered only as exemplary, and not limiting, unless otherwise specifically noted.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited to only those elements but may include other elements not expressly listed or inherent to such process, product, article, or apparatus.

Furthermore, the term “or” as used herein is generally intended to mean “and/or” unless otherwise indicated. For example, a condition A or B is satisfied by any one of the following: A is true (or present) and B is false (or not present), A is false (or not present) and B is true (or present), and both A and B are true (or present). As used herein, including the claims that follow, a term preceded by “a” or “an” (and “the” when antecedent basis is “a” or “an”) includes both singular and plural of such term, unless clearly indicated within the claim otherwise (i.e., that the reference “a” or “an” clearly indicates only the singular or only the plural). Also, as used in the description herein and throughout the claims that follow, the meaning of “in” includes “in” and “on” unless the context clearly dictates otherwise. The scope of the present disclosure should be determined by the following claims and their legal equivalents.

What is claimed is:

1. A method, comprising:
extracting, by a computer from a document, text data describing a patient's visit to a healthcare facility;
removing, by the computer, stop words from the text data, wherein the removing produces clean text data;
determining, by the computer using a large language model, words associated with a medical condition;
filtering, by the computer from the words, fully documented phrases that describe the medical condition, wherein the filtering produces a filtered list of words;
arranging, by the computer, the filtered list of words in order, wherein the arranging produces an ordered set of words;
generating, by the computer using the ordered set of words, a final data set;
training, by the computer, a first machine learning model using the final data set, wherein the first machine learning model is trained to produce a numerical value that indicates a likelihood that the patient's visit to the healthcare facility is related to the medical condition;
determining, by the computer in a pre-processing stage, features particular to the medical condition, each feature having feature attributes derived from historical data including medications, services, and observations pertaining to the medical condition;
preparing, by the computer, a data structure containing the features and the feature attributes;
adding, by the computer, the numerical value from the first machine learning model to the data structure;
applying, by the computer, a second machine learning model on the data structure, wherein the second machine learning model learns from the features, the feature attributes, and the numerical value from the first machine learning model, and generates a patient-specific score indicating a probability of the patient having the medical condition; and
storing, by the computer, the patient-specific score in a data store as evidence of the medical condition being found during the patient's visit to the healthcare facility.

2. The method according to claim 1, wherein the generating the final data set comprises:
tokenizing the ordered set of words into corresponding numerical values;
populating an array with the numerical values; and
padding entries in the array that do not have any numerical values with zeros;

3. The method according to claim 1, wherein the first machine learning model comprises a Convolutional Neural Network model and wherein the second machine learning model comprises an Extreme Gradient Boosting model.

4. The method according to claim 1, wherein the first machine learning model comprises a binary classification model for text processing and wherein the second machine learning model runs an Extreme Gradient Boosting algorithm.

5. The method according to claim 1, wherein the large language model is configured for:
extracting, from the document, document embeddings that describe the document;
extracting, from the document, N-gram words; and
determining, from the document embeddings and the N-gram words using a cosine similarity, the words.

6. The method according to claim 1, wherein the preparing the data structure comprises:
processing a plurality of tables using a configuration file which stores a list of what medications, services, and observations pertain to the medical condition.

7. The method according to claim 6, wherein the configuration file is one of a plurality of configuration files, each specific to a medical condition.

8. A system, comprising:
a processor;
a non-transitory computer-readable medium; and
instructions stored on the non-transitory computer-readable medium and translatable by the processor for:
extracting, from a document, text data describing a patient's visit to a healthcare facility;
removing stop words from the text data, wherein the removing produces clean text data;
determining, using a large language model, words associated with a medical condition;
filtering, from the words, fully documented phrases that describe the medical condition, wherein the filtering produces a filtered list of words;
arranging the filtered list of words in order, wherein the arranging produces an ordered set of words;
generating, using the ordered set of words, a final data set;
training a first machine learning model using the final data set, wherein the first machine learning model is trained to produce a numerical value that indicates a likelihood that the patient's visit to the healthcare facility is related to the medical condition;
determining, in a pre-processing stage, features particular to the medical condition, each feature having feature attributes derived from historical data including medications, services, and observations pertaining to the medical condition;
preparing a data structure containing the features and the feature attributes;
adding the numerical value from the first machine learning model to the data structure;
applying a second machine learning model on the data structure, wherein the second machine learning model learns from the features, the feature attributes, and the numerical value from the first machine learning model, and generates a patient-specific score indicating a probability of the patient having the medical condition; and
storing the patient-specific score in a data store as evidence of the medical condition being found during the patient's visit to the healthcare facility.

9. The system of claim 8, wherein the generating the final data set comprises:
tokenizing the ordered set of words into corresponding numerical values;
populating an array with the numerical values; and
padding entries in the array that do not have any numerical values with zeros;

10. The system of claim 8, wherein the first machine learning model comprises a Convolutional Neural Network model and wherein the second machine learning model comprises an Extreme Gradient Boosting model.

11. The system of claim 8, wherein the first machine learning model comprises a binary classification model for text processing and wherein the second machine learning model runs an Extreme Gradient Boosting algorithm.

12. The system of claim 8, wherein the large language model is configured for:
extracting, from the document, document embeddings that describe the document;
extracting, from the document, N-gram words; and
determining, from the document embeddings and the N-gram words using a cosine similarity, the words.

13. The system of claim 8, wherein the preparing the data structure comprises:
processing a plurality of tables using a configuration file which stores a list of what medications, services, and observations pertain to the medical condition.

14. The system of claim 13, wherein the configuration file is one of a plurality of configuration files, each specific to a medical condition.

15. A computer program product comprising a non-transitory computer-readable medium storing instructions translatable by a processor for:
extracting, from a document, text data describing a patient's visit to a healthcare facility;
removing stop words from the text data, wherein the removing produces clean text data;
determining, using a large language model, words associated with a medical condition;
filtering, from the words, fully documented phrases that describe the medical condition, wherein the filtering produces a filtered list of words;
arranging the filtered list of words in order, wherein the arranging produces an ordered set of words;
generating, using the ordered set of words, a final data set;
training a first machine learning model using the final data set, wherein the first machine learning model is trained to produce a numerical value that indicates a likelihood that the patient's visit to the healthcare facility is related to the medical condition;
determining, in a pre-processing stage, features particular to the medical condition, each feature having feature attributes derived from historical data including medications, services, and observations pertaining to the medical condition;
preparing a data structure containing the features and the feature attributes;
adding the numerical value from the first machine learning model to the data structure;
applying a second machine learning model on the data structure, wherein the second machine learning model learns from the features, the feature attributes, and the numerical value from the first machine learning model, and generates a patient-specific score indicating a probability of the patient having the medical condition; and
storing the patient-specific score in a data store as evidence of the medical condition being found during the patient's visit to the healthcare facility.

16. The computer program product of claim 15, wherein the generating the final data set comprises:
tokenizing the ordered set of words into corresponding numerical values;
populating an array with the numerical values; and
padding entries in the array that do not have any numerical values with zeros;

17. The computer program product of claim 15, wherein the first machine learning model comprises a Convolutional Neural Network model and wherein the second machine learning model comprises an Extreme Gradient Boosting model.

18. The computer program product of claim 15, wherein the first machine learning model comprises a binary classification model for text processing and wherein the second machine learning model runs an Extreme Gradient Boosting algorithm.

19. The computer program product of claim 15, wherein the large language model is configured for:
extracting, from the document, document embeddings that describe the document;
extracting, from the document, N-gram words; and
determining, from the document embeddings and the N-gram words using a cosine similarity, the words.

20. The computer program product of claim 15, wherein the preparing the data structure comprises:
processing a plurality of tables using a configuration file which stores a list of what medications, services, and observations pertain to the medical condition.
